(19) United States
(12) Patent Application Publication
(10) Pub. No.: US 2003/0013633 A1
(43) Pub. Date: Jan. 16, 2003

(54) USE OF MOLECULES THAT MODULATE AN ENERGY RELATED ASSOCIATED STATE

(76) Inventor: RIMA KADDURAH-DAOUK, BELMONT, MA (US)

Correspondence Address: LAHIVE & COCKFIELD, 28 STATE STREET, BOSTON, MA, 02109, US

(21) Appl. No.: 09/327,199

(22) Filed: Jun. 7, 1999

(51) Int. Cl. A61K 31/445

(52) U.S. Cl. 514/1; 514/330

(57) ABSTRACT

The present invention relates to the use of energy kinase modulators for treating or preventing energy related disorders consisting of neurodegenerative disorder, insulin dependent diabetes mellitus and its related disorders, obesity, mitochondrial related diseases and viral infections in a patient experiencing said disorder. The modulators are compounds which can modulate (1) the activity of the enzyme and or its oligomerisation state (2) the nucleotide pool regulated by this kinase (3) the location of the enzyme in the mitochondria or its ability to interact with other mitochondrial components (4) the energy transmitting function of the enzyme and its ability to connect the cytosol and mitochondrial communication and/or (5) flux in and out of the mitochondria.

### RELATED APPLICATIONS

This application claims priority to U.S. Provisional Patent Application Serial No. 60/088,485, filed on Jun. 8, 1998, the entire contents of which are hereby expressly incorporated herein by reference.

This application is related to U.S. Patent Application entitled “Use of Molecules that Modulate the Activity or Function of an Energy Transmitting Kinase, Adenylate Kinase, for Treatment of Energy Related Diseases,” filed concurrently herewith (serial number not yet assigned), U.S. Patent Application entitled “Use of Molecules that Modulate the Activity or Function of an Energy Transmitting Kinase, Glycerol Kinase, for Treatment of Energy Related Diseases,” filed concurrently herewith (serial number not yet assigned), U.S. Patent Application entitled “Use of Molecules that Modulate the Activity or Function of an Energy Transmitting Kinase, Hexokinase, for Treatment of Energy Related Diseases,” filed concurrently herewith (serial number not yet assigned), and U.S. Patent Application entitled “Use of Molecules that Modulate the Activity or Function of an Energy Transmitting Kinase, Nucleoside Diphosphokinase, for Treatment of Energy Related Diseases,” filed concurrently herewith (serial number not yet assigned). This application is also related to U.S. Provisional Application No. 60/088,491, filed on Jun. 8, 1998, U.S. Provisional Application No. 60/088,492, filed on Jun. 8, 1998, U.S. Provisional Application No. 60/088,490, filed on Jun. 8, 1998 and U.S. Provisional Application No. 60/088,489, filed on Jun. 8, 1998. The entire contents, including the background sections and figures, of each of the aforementioned applications are hereby incorporated by reference in their entirety.

### BACKGROUND OF THE INVENTION

The mitochondria is the site of energy production which fuels vital cellular processes. Excitable cells such as muscle, brain and pancreatic beta cells require rapid regeneration of ATP that is mainly provided by oxidative phosphorylation. Oxidative phosphorylation generates the energy needed for building up glycogen and protein stores and is stimulated by metabolites which are utilized in these pathways such as glucose, glycerol, UDP and GDP. Such activation is mediated by specific kinases that are organized at the mitochondrial surface or in between the two mitochondrial membranes and which directly communicate with inner mitochondrial compartment. These kinases transmit information in both directions, cytosol and mitochondria, and transmit information about rate of ATP turnover and substrate level. These kinases are of two groups: energy consuming kinases and energy transmitting kinases (FIG. 1, Brdiczka and Wallimann, Molecular and Cellular Biochemistry 133/134: 69-83, 1994; Brdiczka, Biochimia et. Biophysica Acta 1187, 264-269, 1994). Energy consuming kinases are represented by acyl-CoA synthase (ACS), hexokinase (HK) and glycerol kinase (GK) typically binding to the outer membrane through the mitochondrial voltage sensitive protein porin. The energy transmitting kinases are localized between the inner and the outer mitochondrial membranes and are represented by adenylate kinase, creatine kinase and nucleoside diphosphate kinase. The energy transmitting kinases produce energy rich molecules that can generate ATP and transmit it. Both sets of kinases seem to connect the outer and the inner mitochondrial membranes, and could bind to the adenine nucleotide translocator and porin two proteins involved in the formation of pores such as the mitochondrial transition pore that is believed to be important in the triggering of apoptosis or cell death (Kroemer et. al., Immunology Today 18: 44-51, 1997; Zamzami et.al., J. Exp. Med. 183, 1533-1544,1996; Marchetti et.al., J. Exp. Med. 184, 1155-1160, 1996). Increasing evidence implicates the mitochondria in the regulation of early events of apoptosis (Raff et.al., Science 262, 695-700, 1993; Kroemer, Nature Medicine, 3, 614-620, 1997). The regulation of mitochondrial function, ATP production and mitochondrial transition pore opening may be associated with in the control of cell death of neurons, tumor growth, viral infection, insulin release and other important biological processes.

The adenylate kinase and creatine kinase enzymes are coupled (FIG. 2) with one enzyme potentially replacing the function of the other under certain conditions (Bessman and Carpenter, Annual Reviews of Biochemistry 1985). The adenylate kinase (AK, ATP:AMP phosphotransferase, E.C.2.7.4.3) is a ubiquitous enzyme that catalyses the interconversion of three adenine nucleotides in the cell ; Mg²⁺ ATP+AMP=Mg²⁺ ADP+ADP (Noda, The Enzymes, vol.8, Academic Press, Orlando, Fla., 1973, pp 279305; Atkinson Cellular energy metabolism and its regulation, Academic Press, New York, 1977, pp 85-107). The creatine kinase is an energy generating system operative predominantly in the brain, muscle, heart, retina, and the pancreas. Wallimann et. al., Biochem. J., 281, 21-401 (1992). The components of the system include the enzyme creatine kinase (CK), the substrates creatine (Cr), creatine phosphate (CrP), ATP, ADP, and the creatine transporters. The enzyme reversibly catalyzes the transfer of a phosphoryl group from CrP to ADP to generate ATP. It is found to be localized at sites where rapid rate of ATP replenishment is needed. Some of the functions associated with this system include efficient regeneration of energy in the form of ATP in cells with fluctuating and high energy demand, energy transport to different parts of the cell, phosphoryl transfer activity, ion transport regulation, and involvement in signal transduction pathways.

The regulation of creatine kinase with small molecules has been recently shown to impact the progression of many diseases in animal models. U.S. Pat. No. 5,324,731 entitled “Method of Inhibiting Transformation of Cells in Which Purine Metabolic Enzyme Activity is Elevated”, Kaddurah-Daouk et. al., 1994, describes the antitumor activity of small molecules that regulate creatine kinase. U.S. Pat. No. 5,321,030 entitled “Creatine Analogs Having Antiviral Activity”, by Kaddurah-Daouk et.al., 1994, describes the antiviral activity of small molecules that modulate the creatine kinase system. U.S. Pat. No. 5,676,978 “Methods of Inhibiting Undesirable Cell Growth Using a Combination of Creatine Compounds and a Hyperplastic Inhibition Agent”, by Teicher et. al., 1997, describes the beneficial effect of using creatine kinase modulators along with standard cancer chemotherapeutic agents for the treatment of solid tumors. Modulators of creatine kinase were also shown to protect neurons and inhibit progression of several neurodegenerative diseases (Matthews et. al., J Neurochemisty Jan, 1998).

### SUMMARY OF THE INVENTION

The present invention provides methods of treating an energy related associated state by administering to a subject an effective amount of an energy kinase modulator, such that an energy related associated state is treated. These modulators could modify an energy related associated state by modifying (1) the activity of enzymes, e.g., nucleoside diphosphokinases, adenylate kinases, glycerol kinases, hexokinase, or creatine kinase, or their oligomerisation state (2) the nucleotide pool regulated by these kinases (3) the location of the enzymes in the mitochondria or their ability to interact with other mitochondrial components (4) the energy transmitting function of the enzymes and their ability to connect the cytosol and mitochondrial communication and/or (5) flux in and out of the mitochondria.

The invention further pertains to methods for modulating an energy transmitting kinase in a subject by administering to a subject an effective amount of an energy transmitting kinase modulator, such that an energy transmitting kinase is modulated.

The invention further pertains to methods for treating an energy related associated state, comprising administering to a subject an effective amount of an energy kinase modulator, such that an energy related associated state is treated, provided said energy related associated state does not involve creatine kinase.

Compounds that can be used in one of the aforementioned methods will be generated by screening random or biased combinatorial libraries based on the nature of the substrates of the enzymes and their mechanism of action. Alternatively these compounds could be generated using rational approaches such as drug design using the known x ray structure of the enzymes when available.

Examples of such compounds, modulators, include but are not limited to bepridil, verapamil, nifedipine, nisoldipine, bronchodilators, e.g. theophylline, semecarpus anacardium nut extract, 2-dioxy-D-glucose, antioxidants, e.g. vitamin E, purines, such as caffeine or theophylline, or dipyridamole, papaverine, or cyclamidomycin (desdanine).

Disorders that can be treated are selected from the group consisting of neurodegenerative disorders, insulin dependent diabetes mellitus and its related disorders, obesity, mitochondrial related diseases and viral infections. The invention comprises administering to a subject afflicted with or susceptible to said disorder an amount of a modulator (compounds which modulate one or more of the structural or functional components of the energy kinase system) sufficient to alleviate or prevent the symptoms of the disorder. The modulator may be in the form of a pharmacologically acceptable salt or combined with an adjuvant or other pharmaceutical agent effective to treat or prevent the disease or condition.

Prior to the present invention, the above referenced kinase systems had not been directly implicated as a target for drug design for the generation of molecules that are of therapeutic value for the treatment or prevention of the above mentioned diseases. The structural data available for the enzyme along with the enzymatic reaction and mechanism of action of the enzyme and the identified substrates will facilitate the screen and design of small molecules that modify the activity or function of the enzyme system and hence modify disease progression.

The present invention also provides pharmaceutical compositions containing the modulators in combination with a pharmaceutically acceptable carrier. The present compositions may be used in combination with effective amounts of standard chemotherapeutic agents.

Packaged drugs for treating subjects having a disease relating to energy related states referred to above also are the subject of the present invention. The packaged drugs include a container holding the adenylate kinase compound, in combination with a pharmaceutically acceptable carrier, along with instructions for administering the same for the purpose of preventing, ameliorating, arresting or eliminating a disease.

By treatment is meant the amelioration of one or more symptoms of, or total avoidance of, the disorder as described herein. By prevention is meant the avoidance of a currently recognized disease state, as described herein, in a patient evidencing some or all of the symptoms of the disorders described above. The present compositions may be administered in a sustained release formulation. By sustained release is meant a formulation in which the drug becomes biologically available to the patient at a measured rate over a prolonged period. Such compositions are well known in the art.

It is an object of the present invention to provide methods for preventing or treating diseases that relate to energy related associated states(diseases that have mitochondrial dysfunctions or defects in the transmission of metabolic state between cytosol and mitochondria) by administering to an afflicted individual an amount of a compound or compounds, e.g., modulators, which modulate one or more of the structural or functional components of the above referenced kinases associated with an energy related associated state sufficient to prevent, reduce or ameliorate the symptoms of the disease. These compounds are collectively referred to as “modulators”.

### BRIEF DESCRIPTION OF THE FIGURES

FIG. 1: Describes the location of energy transmitting and energy consuming kinases in the mitochondria. It shows the organization and function of several kinases at the mitochondrial surface. One group of kinases, named energy consuming kinases, are bound to the outer membrane pore protein: acyl-CoA synthase (ACS), hexokinase (HK), and glycerol kinase (GK). A second group named “energy transferring” kinases are located between the two envelope membranes: adenylate kinase (ADK), mitochondrial creatine kinase (mCK), and nucleoside diphosphate kinase (NuDiKi). Some kinases (HK, GK, mCK, NuDiKi), which were enriched in the contact site fraction, are thought to form complexes with the outer membrane pore (P) and the adenine nucleotide translocator (AT). To keep the scheme clear, the reactions of several kinases are shown in abbreviated detail.

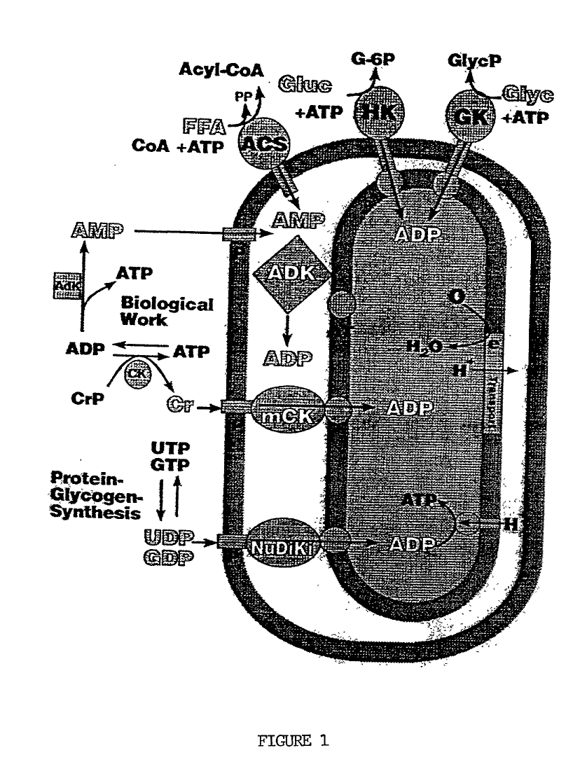

FIGS. 2a and 2b: The coupling of the adenylate kinase and creatine kinase reactions. FIG. 2a shows the arrangement of the three enzymes, the two adenylate kinase molecules and one creatine kinase around the site of oxidative phosphorylation to produce a common pool of ADP and sites for entry for ATP, creatine-creatinephosphate, and AMP. FIG. 2b shows the sequential arrangement of adenylate kinase molecules to maintain substrate flow between creatine kinase and myosin ATPase.

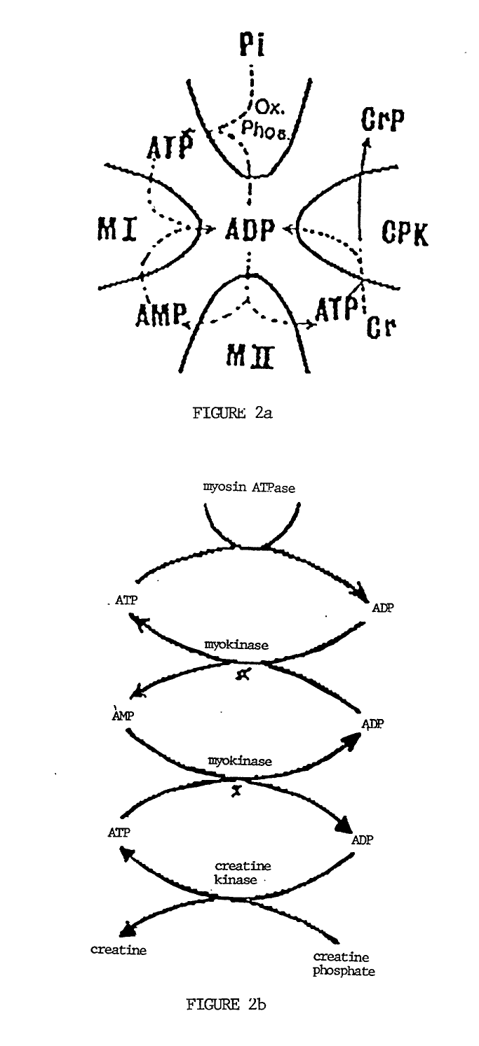

FIG. 3: Glycerol Kinase. Structure of phosphoryl group acceptors utilized by glycerol kinase.

### DETAILED DESCRIPTION OF THE INVENTION

The features and other details of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention provides methods of treating an energy related associated state by administering to a subject an effective amount of an energy kinase modulator, such that an energy related associated state is treated.

The invention also pertains to methods for modulating an energy transmitting kinase in a subject by administering to a subject an effective amount of an energy transmitting kinase modulator, such that an energy transmitting kinase is modulated.

The invention further pertains to methods for treating an energy related associated state, comprising administering to a subject an effective amount of an energy kinase modulator, such that an energy related associated state is treated, provided said energy related associated state does not involve creatine kinase.

In one embodiment, the invention pertains to methods for treating an energy related state which does not involve the direct modulation of creatine kinase by the administered energy kinase modulator. The method involves administering to a subject an effective amount of an energy kinase modulator such that the energy related state is treated, provided that it does not involve the direct modulation of creatine kinase by the administered energy kinase modulator.

Components of the energy related states which can be modulated include the enzymes such as adenylate kinase, glycerol kinase, hexokinase or nucleoside diphosphokinase, the substrates, the association of these kinases with other components mainly protein and lipids.

As used herein, the term “modulate” means to change, affect or interfere with the functioning of the components of these kinase enzyme systems, e.g., the enzymatic activity. For example, the modulation can be the inhibition of or enhancement of enzymatic activity.

The phrase “energy related state” is intended to include diseases or conditions which involve the use of energy, e.g., the transmission of, impairment of or consumption of energy. Suitable examples of energy related states include neurodegenerative disorders, insulin dependent diabetes mellitus and its related disorders, obesity, mitochondrial related diseases, cancer and viral infections.

The term “energy kinase modulator” is intended to include molecules which can modulate a targeted kinase system, e.g., effect the ability of a kinase to transmit, impair or consume energy, e.g., energy associated with mitchondrial activity. Suitable examples of modulators include those modulators art-recognized for the decribed enzymatic systems and those described in the related copending applications.

The term “state associated with energy impairment” is intended to include those states which impair, e.g., decrease or eliminate, the transmission of energy through, for example, mitochondria. States which are typically associated with energy impairment include, but are not limited to, neurodegenerative disorders, insulin dependent diabetes mellitus and its related disorders, obesity, mitochondrial related diseases, cancer and viral infections.

The phrase “modulation of an energy transmitting kinase” is intended to include changes to or effects on a targeted kinase system that results in a change in the kinase system which leads to a biologically beneficial effect in a subject being treated for an energy related state, e.g., effects the ability of a kinase to transmit, impair or consume energy, e.g., energy associated with mitchondrial activity. The phrase is intended to include an increase, decrease, or elimination of the activity of an energy transmitting kinase. Preferably this modulation is effected such that a particular targeted aberrant disease or condition is controlled while other cells which are not detrimental to the subject are allowed to remain substantially uncontrolled or substantially unaffected. Representative examples of energy transmitting kinases include adenylate kinases, glycerol kinases, hexokinases, creative kinase and nucleoside diphosphokinases. In certain embodiments of the invention, creatine kinase is not included as a member of energy transmitting kinases.

The Adenylate Kinase System:

The adenylate kinase (AK, ATP: AMP phosphotransferase, E.C.2.7.4.3) is a ubiquitous enzyme that catalyses the interconversion of three adenine nucleotides in the cell; Mg²⁺ ATP+AMP=Mg²⁺ ADP+ADP (Noda, The Enzymes, vol. 8, Academic Press, Orlando, Fla., 1973, pp 279-305; Atkinson Cellular energy metabolism and its regulation, Academic Press, New York, 1977, pp 85-107). In vertebrates, three isozymes (AK1, AK2 and AK3) have been identified to date. AK1 is present in the cytosol of skeletal muscle, brain and erythrocytes, while AK2 exists both in the cytosol and the mitochondrial intermembrane space of liver, kidney, spleen and heart (Khoo, Biochim. Biophys. Acta 268, 98-101, 1972). AK3, GTP: AMP phosphotransferase is localized exclusively in the mitochondrial matrix of various tissues (Tomasselli et. al., Eur. J. Biochem. 93, 257-262, 1979). Although AK is thought to contribute to homeostasis of the adenine nucleotide composition in the cell, the precise role of the AK isozymes is yet unknown. Among the AK isozymes, the AK2 type enzyme is distributed in a number of organisms from prokaryotes to eukaryotes and the genetic background of this enzyme has been investigated (Nakazawa et. al., Progress in Clinical Biological Research vol. 344, Wiley-Liss New York, 495-514, 1990; Tomasselli et. al., Eur. J. Biochem. 155, 111-119, 1986; Brune et.al., Nucleic Acids Res. 7139-7151, 1985; Kishi et. al., J. Biol. Chem. 262, 11785-11789, 1987; Tanabe et. al., J. Biochem. Tokyo 113, 200-207, 1993; Tanaka et. al., Gene 93, 221-227, 1990). Two subtypes of AK2 have been identified, AK2A and AK2B, that differ in the C terminal portion. The cDNA of the AK2 genes have been reported (Lee et.al., Biochem. Mol. Biol. Int. 39, 833-842, 1996; Noma et. al., Biochimica et. Biophysica Acta 1395, 34-39, 1998). Northern blot analysis demonstrated that AK2 mRNA is strongly expressed in liver, heart, skeletal muscle and pancreas, and moderately in kidney, placenta and brain with week expression in the lung. However, Western blot analysis showed that the protein is present in large amounts in liver, heart, kidney and in small amount in lung and undetectable in brain and skeletal muscle. These results suggested the presence of tissue-specific gene expression including post translational regulation in expression of the Ak2 gene.

In general the adenylate kinase system is active in tissues that have high energy turn over from the adenine nucleotide pools. It is believed that one important function of the AK system is its involvement in the maintenance of equilibrium among the adenine nucleotides, thereby functioning in the maintenance of energy charge. Since AK is localized in cell compartments, is itself subject to control, and affects adenine nucleotide concentrations which serve in turn to exert metabolic control making this enzyme system an important one for energy economy of living systems.

The enzyme from rabbit muscle was isolated and crystallized (Noda and Kuby JBC, 226, 541, 1957), physical properties and kinetics have been studied (Noda and Kuby JBC, 226, 551, 1957; Noda, JBC 232, 237, 1958). Other purification and properties of the enzyme from the same source have been reported (Callaghan BJ 67, 651, 1957; Callaghan and Weber BJ73, 473, 1959). The bovine mitochondrial form has been extensively studied (Markland et. al., JBC 241, 4124, 1966; Markland et. al., JBC 241, 4136, 1966). Brdiczka (Brdiczka et. al., Eur. J. Biochem. 5, 294, 1968) demonstrated that the mitochondrial enzyme in rat liver is localized between the inner and the outer mitochondrial membranes. Criss (Criss et. al., J. Biochem. Tokyo 70, 273, 1971) observed that phosphate as well as alkaline pH promote flux for AK to the outside of the mitochondria, and that while nucleotides did not prevent, the addition of divalent metals did prevent the phosphate-induced release. The release of the enzyme slightly preceded or accompanied mitochondrial swelling. The mitochondrial uptake of released AK under conditions of initial, but not complete, swelling was reversible to an extent by the addition of ADP (1 mM), MgCl2 (3 mM), and glutamate (2 mM). Like the finding of the release of many enzymes from the mitochondria under physiological conditions, the reported flux of AK across the outer mitochondrial membrane is of great interest to the understanding of the intimate mechanisms of energy metabolism in the cell.

The high energy phosphate transferring ability of the enzyme results in energy charging and transport and in production of ADP which is channeled to the mitochondria for the control of oxidative phosphorylation and mitochondrial function. The adenine nucleotide pool controlled partially by this enzyme system provides the cell with metabolic control. The metabolic regulation by this enzyme system could be achieved by several ways including modulating (1) the activity of the enzyme (2) the adenine nucleotide pool regulated by this kinase (3)the location of the enzyme in the mitochondria or its ability to interact with other mitochondrial components (4)the energy transmitting function of the enzyme and its ability to connect the cytosol and mitochondrial communication and/or (5) flux in and out of the mitochondria.

Adelman (Adelman et. al., JBC 243, 2538, 1968 ) found that the activity of adenylate kinase in rat liver was influenced by diet and hormones. Fasting induced an increase in activity while refeeding a high glucose diet dropped the level. In Alloxan diabetic rats the level of the enzyme was high and dropped upon treatment with insulin. The enzyme system was suggested to be implicated in gluconeogenesis. Several isoforms of adenylate kinase were shown to be developmentally regulated (Filler et. al., BJ 122, 553, 1971; Hommes et. al., BBA 230, 327,1971; Kendrick-Jones et. al., BJ 103, 207, 1967).

Enzymatic Assays:

The most generally reliable assay, applicable under the widest possible variation of conditions are methods dependent upon measuring changes in the amounts of the adenine nucleotides brought about in a measured interval of time by adenylate kinase as determined after separation of the nucleotides by chromatographic procedures. An example of such a method has been the use of Dowex-1 resin with electrophoretic techniques using thin layer chromatography and chromatography paper (Sato et. al., Anal Biochem. 5, 542, ; Randerath, Nature, 194, 768, 1962; Krebs et. al., BBA 12, 172, 1953).

Spectroscopic procedures involve measuring a reaction catalyzed in the direction of ATP formation and coupling to other enzymes such as hexokinase with excess glucose and glucose-6-phosphate dehydrogenase together with TPN, or by coupling to creatine kinase. In the case of creatine kinase excess creatine is added together with creatine kinase to yield creatine phosphate from the ATP formed by adenylate kinase. Another widely used coupled reaction is the use of adenylate kinase and ATP coupled with excess phosphoenolpyruvate together with lactate dehydrogenase and excess DPNH. The decrease in DPNH is measured by the decrease in absorbence at 340 nm with time (Oliver BJ 61, 116,1955; Adams Biochem. Z. 335, 25,1961).

A pH-stat assay couples the reaction of adenylate kinase with ADP as substrate with the hexokinase reaction carried out at pH 8, in which one mole of hydrogen ions is released for every mole of ATP formed by adenylate kinase. The rate to which standardized alkali is added to maintain the pH is a measure of adenylate kinase activity.

An alternative approach for determining enzyme activity is the use of labeled substrates that can be separated and visualized on sensitive films.

The Glycerol Kinase System:

Glycerol kinase catalyzes the stereospecific transfer of the terminal phosphoryl moiety of ATP to one of the primary hydroxyl groups of glycerol, forming sn-glycerol 3-phosphate.

The glycerol kinase also referred to as ATP: glycerol phosphotransferase (EC 2.7.1.30) or GK and glycerate kinase ATP: D-glycerate 3 -phosphotransferase (EC 2.7.1.31) are enzymes that catalyze the transfer of the terminal phosphate of ATP to glycerol and D-glycerate respectively according to the following equations:

Glycerol+ATP=sn glycerol 3-phosphate+ADP

D-glycerate+ATP=3-phospho-D-glycerate+ADP

For review see Thomer and Paulus, The Enzymes, Academic Press, Orlando, Fla., vol 8 487-508, 1973. These enzymes could acquire different properties depending on the source.

Metabolic Role:

The glycerol 3 phosphate has diverse metabolic fates. It is an intermediate in the catabolism of glycerol; it serves as an intermediate for triglycerides and complex lipids as well as for macromolecules such as teichoic acids; and it participates in cycles of reactions by which electrons can be transferred from cytoplasm into mitochondria. The function of glycerol kinase might differ in different tissues.

In higher organisms the primary role of glycerol kinase seems to be the salvage of glycerol released upon lipolysis. This is demonstrated in the mammalian intestines and the brown adipose tissue in rats where glycerol produced by lipolysis is rephosphorylated by high levels of glycerol kinase and reused for lipid synthesis. In the white adipose tissue glycerol kinase levels are very low; presumably glycerol released in this tissue as well as that produced in the capillaries by lipoprotein lipase, is reesterified in the liver and kidney which has a high content of glycerol kinase. Genetically obese animals have abnormally high levels of the enzyme (Koschinsky et.al., Biabetologia 7, 316, 1971; Treble and Mayer Nature, 200, 363, 1963; Koschinsky and Gries HoppeSeyler's Z. Physiol. Chem. 352, 430, 1971). In tissues where reducing power is not extensively used for biosynthesis, cytoplasmic dihydroxyacetone-P is reduced by a NAD-link glycerol 3 phosphate dehydrogenase to glycerol 3 phosphate, which can enter the mitochondrial inner membrane space to be reoxidized by flavin linked enzymes (Racker, Mechanisms in Bioenergetics p 100, Academic Press, 1965; Holohan et.al., Fed. Proc., Fed Amer. Soc. Exp. Biol. 31, 421, 1972). The net result of this cycle is transfer of electrons from the cytoplasm into the mitochondria, but although glycerol 3 phosphate participates catalytically, it will occasionally have to be replenished.

Molecular Properties:

The molecular weight of this enzyme was determined by equilibrium sedimentation to be 217,000. Equilibrium ultracentrifugation and polyacrylamide gel electrophoresis techniques revealed a single molecular species of 56,00 molecular weight which suggests that glycerol kinase is a tetrameric protein composed of four similar or identical sub units.

Catalytic Properties and Substrates:

Glycerol kinase catalyzes the stereospecifric transfer of the terminal phosphoryl moiety of ATP to one of the primary hydroxl groups of glycerol, forming sn-glycerol 3-phosphate.

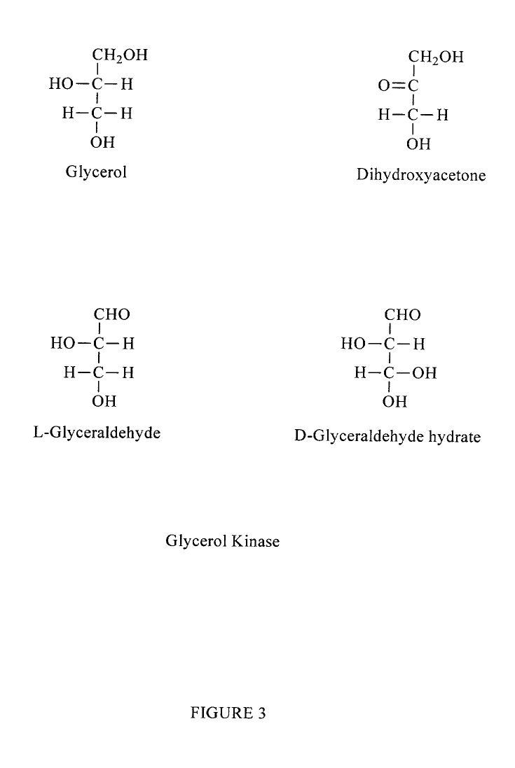

The enzyme can catalyze the phosphorylation of dihydroxyacetone, L-glycerolaldehyde and D-glyceraldehyde hydrate (FIG. 3) The affinity for these compounds is much less than that for glycerol; on the other hand the phosphorylation of dihydroxyacetone is often more rapid.

The phosphoryl donor used in the GK reaction is typically ATP. The bacterial enzyme can only utilize ATP and exhibits the highest degree of specificity. GK from brown fat is reported to utilize CTP even better than ATP, and the enzyme from rat heart and white fat utilize equally UTP and ATP (Treble and Ball, Fed. Proc., Fed. Amer. Soc. Exp. Biol. 22, 357, 1963; Robinson and Newsholme B. J. 104, 2C, 1967; Hayashi and Lin J. B. C. 242, 1030, 1967). AMP is an uncompetitive inhibitor for the enzyme and the products of the reaction ADP and glycerol -3-phosphate are also inhibitors for the enzyme. The inhibition by glycerol 3 phosphate is completely eliminated by low concentrations of phosphate or sulphate ions suggesting an allosteric mechanism. The glycerol kinases of several bacterial species are specifically inhibited by fructose 1, 6-diphosphate (non competitive) but not the human enzyme.

Enzymatic Assays:

The activity of glycerol kinases is found in different tissues and organisms. Examples of tissue include liver, kidney, intestine, muscle, fat, mammary gland and sperm. The enzyme assay could be based on the amount of product produced where glycerol 3 phosphate is coupled to reactions that can result in the reduction of NAD+ in the presence of glycerol 3 phosphate dehydrogenase. This could be monitored spectrophotometrically. The reactions in these assays could be done in parallel or sequentially where the GK reaction and the other coupled enzymes could be added in parallel or alternatively where the GK reaction is allowed to go to completion and then the other coupled enzymes are added (Wieland and Suyter, Biochem Z 329, 320, 1957; Ublitz and Yieland methods of Enzymology 5, chapter 46, 1962; Bublitz and Kennedy JBC, 211, 951-963, 1954). Alternatively the reaction can be followed by coupling ADP formation to the oxidation of NADH in the presense of PEP, pyruvate kinase and lactate dehydrogenase (Garland and Randle, Nature, 196, 987, 1962).

Radiochemical assays have also been developed and have higher sensitivity and greater flexibility than the spectrophotometric procedures. The formation of glycerol 3 phosphate from radioactive glycerol can be measured by chromatographic separation, by precipitation, or by absorption to DEAE filter paper (McBride and Korn J Lipid Res. 5, 442, 1964; Hayashi and Lin BBA 94, 279, 1965; Newsholme et.al., BBA, 132, 338, 1967).

Alternatively, the glycerol dependent conversion of gamma 32P labeled ATP to an acid stable phosphate ester can be measured (Thormer and Paulus JBC, 246, 3885, 1971).

In the preparation of the enzyme from different tissues it became clear that although most of it is soluble some percent is associated with particulate material especially in mitochondrial membranes.

The Hexokinase System:

The hexokinases catalyze the phosphorylation of glucose by ATP as the first step in the utilization of glucose and occurs in all eukaryotic cells. Prokaryotic cells also use this enzyme system. Glucose 6 phosphate, the end product of the reaction, could be utilized in different ways dependent on cell type. Meyerhof was among the early investigators to study the yeast enzyme (Meyerhof, Biochem. Z 183, 176, 1927). Extensive studies exist on the kinetics, structure and genetics of the hexokinases ( Colowick, In P. D. Boyer, ed, The Enzymes, 3rd edition, 9 B, Academic Press, New York, 1-48, 1973; Ureta, Comp. Biochem. Physiol. B71, 549, 1982; Lobo and Maitra Genetics 86, 724, 1997; Wilson in R. Beitner ed. Regulation of Carbohydrate Metabolism 1, CRC Press, Boca Raton, Fl. 45-85, 1985; Ureta and Medina, Arch. Biol. Med. Exp. 20, 343, 1987; Middleton, Biochem. Soc. Trans. 18, 180, 1990; Wilson, Rev. Physiol. Biochem. Pharmacol. 124, 65, 1995).

Glucose is the preferred substrate of the hexokinases but they can phosphorylate other hexoses to varying degrees as recognized by the name hexokinase (ATP; D-hexose 6-phosphotransferase, E C 2.7.1. 1). Only a few hexokinases are glucokinase specific. Hexokinases from different species have different molecular weight and distribution and typically there is a mixture of isoenzymes. The initial work done was on yeast hexokinases. There are at least three human isozymes; PI (A), PII (B) and glucokinase. In animals initial enzyme isolation was from liver. In vertebrates there are at least four isozymes, hexokinases A,B,C,D or alternatively I,II,III,IV, classified on the basis of electrophoresis mobility. The D or IV is called glucokinase. Type I is found in brain and kidney as well as heart and intestine, type II is found mainly in skeletal muscle, fat pads, heart and intestine, type III is found in the kidney and intestines, while liver contain all four types. Type III has a low Km and is inhibited by excess glucose, while type IV requires high glucose concentration for detection. Many of these hexokinases are hormonally and glucose regulated. Type IV enzyme in the liver undergoes tight regulation with hormonal changes. Type II appears to decrease with starvation or diabetes, and to reappear with refeeding or insulin administration. This isozyme is found in insulin sensitive tissues. In man and rodents Hexokinase IV appears to play a major role in insulin secretion acting as a glucose sensor.

Types I, II,and III have many features in common, tight inhibition by glucose 6 phosphate which is competitive with ATP but not glucose, and moderate inhibition by ADP which is not inhibited by either ATP or glucose. Nucleotide specificity is similar for all three types, with ATP giving by far the highest activity and ITP giving appreciable activity in all cases. Molecular weights of the hexokinase are around 96,000 to 100,000.

The vertebrate hexokinases typically act on mannose, fructose and 2 deoxy glucose with glucose being the preferred substrate.

Certain hexokinases have been suggested to be involved in the transport of glucose into cells possibly via the regulation of the transporter. There is a correlation between the expression of certain hexokinase isoenzymes and different transporters of glucose (Cardens et al., Biochemica. et. Biophysica. Acta 1401, 242-264, 1998). A question is raised whether the hexokinase isoenzymes and the glucose transporters may be homologous, with associations between particular isoenzymes and particular transporters to form functional units, with close physical interaction, that has co evolved.

Eukaryotic hexokinases prefer ATP as the nucleotide substrate, while bacterial enzymes seem less specific and ITP is a relatively good substrate.

Most native hexokinases show molecular masses of 50 or 100 kDa. Gene duplication and gene fusions might have been responsible for the higher molecular masses of the hexokinases. New acquired functions might have also evolved with the change in mass example being evolvement of new sites facilitating inhibition by glucose 6 phosphate.

Some of the hexokinase genes have more than one promoter with tissue specific expression.

It has been recognized since the work of Crane and Sols (Methods of Enzymology vol 1, 277, 1955; JBC, 210, 597, 1954) that a considerable fraction of total hexokinase activity in tissue homogenates is associated with particles. Katzen and colleagues (Katzen et. al JBC vol 245, 4081, 1970) have shown that the hexokinases found bound to the mitochondria or microsomal fractions are indistinguishable from types I and II found in the soluble fraction, and that type III dose not occur in the particulate form. The bound form is present in a latent form which becomes available to added substrates only upon destruction of the membrane structure. It is now generally agreed that the hexokinases in tissue particles are largely associated with mitochondria. The enzyme associates with the voltage sensitive protein porin. The specific loading of the mitochondria with calcium phosphate during oxidative phosphorylation resulted in the predictive change in the density of the particles bearing the hexokinase activity. The level of bound enzyme to free enzyme depends on the concentration of substrates and products as well as ions such as Mg, Mn or Ca. High salt concentration and low pH favor the elution of the enzyme from the mitochondria.

The N terminal portion of Hexokinase A might modulate the binding of the enzyme to the mitochondria. There is a highly conserved dodeca peptide at the begging of the N terminal sequence responsible for the interaction with porin (Arora et.al., J. Bio. Chem. 268, 18259, 1993). There is also a binding site for glucose 6 phosphate that controls the binding of the brain enzyme to mitochondria, regulating the relative levels of soluble and membrane bound forms. The presence of this effector determines the association of the hexokinase from the membrane. Arora (Arora et.al., JBC, 268, 18259,1993) proposed that the N terminal half might serve as a spacer between the membrane and the catalytic C terminal half of the enzyme allowing this to interact easily with the next glycolytic enzyme, i.e., phosphoglucose isomerase.

In the mammalian liver, it was noted that the liver contained a high and a low Km activities of hexokinases and that the high Km enzyme disappeared preferentially during starvation or diabetes, and reappeared during refeeding or insulin administration (Vinuela JBC 238, PC 1175,1963; Sharma et al., JBC 238, 3840, 1963; Niemeyer et.al., Nature 198, 1096, 1963; Salas et.al., JBC, 238, 3535, 1963). The high Km enzyme is restricted to hepatocytes where as the bulk of the low Km activity is associated with non parenchymal tissue of the liver.

The yeast enzyme exists as a dimer with subunit molecular weight of 50,000. Type I mammalian enzyme have no subunits but consists of a single chain of molecular weight 100,000.

There is a tendency of type one hexokinase to undergo aggregation during sedimentation equilibrium ultracentrifugation.

Enzyme Mechanism:

There is evidence that the mammalian enzymes like yeast enzymes, can form binary complexes with glucose much more readily than with ATP. Glucose can prevent inactivation of mammalian enzymes type II and IV by proteolytic enzymes and of type I by Ellman's reagents. Mg ATP had no protective effect. It seems that Mg ATP does not bind readily to the mammalian enzymes unless glucose in present. Substrate addition seems to be ordered with glucose addition preceding ATP addition in the reaction sequence.

A phosphoenzyme intermediate has been suggested but not proven, and a sequential mechanism of addition has been partially validated.

Regulation of Activity:

Stricking inhibition by glucose 6 phosphate. This seems to be at a regulatory site rather than at the catalytic site (allosteric control). ADP also inhibits the enzyme with evidence of doing so at the catalytic site. The inhibition of the enzyme by glucose 6 phosphate can be attenuated by inorganic phosphate. Citrate and catecholamines stimulate type I hexokinase under low pH conditions. Mixed inhibition by ADP, strong inhibition by N-acylated derivatives of glucosamine, broad sugar specificity, and narrow nucleotide specificity towards ATP among the natural nucleoside triphosphates.

Inhibition by Glucose:

A,B,D studied, C is inhibited by excess glucose.

The NDP Kinase System:

The term “nucleoside diphosphokinase” (NDP kinase, ATP: nucleoside diphosphate phosphotransferase, EC 2.7.4.6.) is used to designate a family of relatively unspecific enzymes that catalyze the transfer of the terminal phosphate group of 5′-triphosphate nucleotides to 5′-diphosphate nucleotides by the following general mechanism: N₁TP+N₂DP=N₁DP+N₂TP.

Where N₁ and N₂ are purine or pyrimidine ribo or deoxyribonucleosides. All NDP kinases that have been examined to date function through the formation of enzymebound high energy phosphate intermediates. The first NDP kinases were identified from yeast and pigeon muscle in 1953 by Berg, Nature 172, 1008, 1953 nad Krebs, BBA 12, 172, 1953. The specificity of the NDP kinases is still controversial and the possibility of subspecies of kinases with greater specificity for specific nucleosides is still a possibility.

In a detailed study with the crystalline yeast enzyme Garces and Cleland found that the overall equilibrium constant at pH 8.0 is 1.28. However the partial equilibrium constant for the phosphorylation of free enzyme by ATP is 0.188, and for dephosphorylation of the phosphorylated enzyme by UDP it is 6.76 as shown by the following equations:

E+ATP=E−P+ADP (Keq. 0.188)

E−P+UDP=E+UTP (Keq. 6.76)

These equilibria favor the free enzyme and suggest that the phosphorylated enzyme has a free energy of hydrolysis about 1.0 kcal more negative than the terminal phosphate bond of ATP (Garces Biochemistry 8, 633, 1969).

The first crystallized enzyme was extracted from yeast by Ratliff JBC, 239, 301, 1964. The enzyme was later crystallized from other species. Studying the mechanism of action of the enzyme revealed a ping pong reaction sequence and functions through the formation of a stable phosphate bond with the enzyme as determined by labeling experiments (Mourad, JBC, 241, 271, 1966; Mourad, BBRC, 19, 312, 1965, Mourad JBC 241, 3838, 1966). All NDP kinases seem to function by similar reaction mechanisms. The phosphorylated amino acid seems to be the Histidine residue. Upon electrophoresis multi enzyme species were identified.

The enzyme was found to be ubiquitously distributed in nature and is found in animals plants and microorganisms (Parks and Agrawal, The Enzymes, vol. 8, Academic Press, Orlando, Fla., 1973, pp 307-333). The human erythrocytes express abundant activity of the enzyme. The existence of several activity peaks suggested that these enzymes might not be isoenzymes but rather a family of related enzymes functioning through high energy phosphate intermediates.

Most of the isolated kinases have molecular weights in the range of 80,000 110,000 daltons. The number of phosphate binding sites per mole of protein suggests that the enzyme might exist as a tetramer (Garces and Cleland, Biochemistry, 8, 633, 1969). Other oligomerisation states have been identified.

All NDP kinases studied suggest the essential role of sulfhydryl groups. The enzymes are inhibited by mercurial reagents such as p-chloromecuribenzoate, and reactivated by the addition of thiols.

The NDP kinases are major components of the cell for the synthesis of nucleotide triphosphates and hence has a major impact on cellular energy homeostasis. The activity of these kinases is tissue is very high 10-100 fold greater than the activity of the monophosphate nucleotide kinases. The specificity of the NDP kinases seems to be less stringent than the monokinases. A role of these kinases in mitochondrial oxidative phosphorylation, membrane transport involving ATPases, and mitochondrial cytoplasm communication has been suggested.

Enzymatic Assays:

Various procedures have been developed for the measurement of NDP kinase activity. They fall into three major groups: (1) coupled enzymatic assays, (2) isotopic assays, (3) staining procedures for localizing NDP kinase activity after chromatography or electrophoresis (Parks and Agrawal, The Enzymes, vol.8, Academic Press, Orlando, Fla., 1973, pp 307-333).

(1)-Coupled Enzymatic Reactions

Several coupled enzymatic procedures have been developed in which the reaction rate is measured spectrophotometrically by measuring the appearance or disappearance of NADH or NADPH. These assays enable dynamic measurements, are sensitive, rapid and easy to operate. Two popular assays are given here as examples:

a—Pyruvate Kinase-Lactate Dehydrogenase Method: This procedure follows the formation of ADP from ATP or GDP from GTP in a coupled reaction system containing phosphoenolpyruvate (PEP), NADH, pyruvate kinase, lactate dehydrogenase and the nucleotide substrates plus the appropriate salts and buffers according to the following reactions:

dGDP+ATP=dGTP+ADP

ADP+PEP=ATP+pyruvate

pyruvate+NADH=lactate+NAD

The rate of decrease in absorbance at 340 nm is followed spectrophotometrically.

b—Hexokinase-Glucose-6-Phosphate Dehydrogenase Assay: The assay is based on the formation of ATP from ADP and ITP or UTP in the presence of hexokinaseglucose-6-phosphate dehydrogenase indicator system according to the following equation:

ITP+ADP=IDP+ATP

ATP+glucose - - - glucose 6-phosphate+ADP

glucose 6-phosphate+NADP - - - 6-phosphogluconate+NADPH+H⁺

Variations around this assay system have been developed where other coupling enzyme systems were used.

(2)-Isotopic Procedures:

One of the most commonly used procedure for NDP kinase assays is the ATPADP exchange or a related isotope method (Chiga and Plaut, JBC 234, 3059, 1959). A C14 labeled diphosphate nucleotide is incubated with nonlabeled triphosphate nucleotide. The products are separated using chromatography techniques and the level of incorporation of radioactivity in the triphosphate nucleotide is determined. A variety of isotopes could be used instead of C14, example would be P32 radioisotope.

(3)-Staining Procedure for NDP Kinases:

A convenient staining procedure has been developed for the identification of NDP kinase activity. Bands following gel electrophoresis chromatography are identified. This method is similar to the coupled hexokinase reaction mentioned above with the exception that the NADPH formed is detected by reaction with tetrazolium ( Cheng Biochemistry 10, 1971). A useful method has been described were the complete identification system is incorporated into agarose overlay which becomes stained using the NDP kinase reaction (Cheng Biochemistry 10, 1971). Since this staining method usually employs UTP and ADP as substrates, it is necessary to run coreactions for contaminating adenylate kinase by omitting UTP.

A modulator is a compound that modifies (1) the activity of the enzyme and or its oligomerisation state (2) the nucleotide pool regulated by this kinase (3) the location of the enzyme in the mitochondria or its ability to interact with other mitochondrial components (4) the energy transmitting function of the enzyme and its ability to connect the cytosol and mitochondrial communication (5) fluxes in and out of the mitochondria.

Existing or specially designed combinatorial libraries will be screened for molecules that modulate the NDP kinase system. Alternatively compounds could be rationally designed based on the know x ray structural information and the mechanism of action. Compounds of therapeutic interest could be identified as those that affect the enzymatic activity of NDP kinase, its oligomeric state, nucleotide pools, its association with mitochondrial components, it's effect on mitochondrial function among other parameters.

For identifying compounds that affect the enzymatic activity of NDP kinase the above referred to enzymatic assay reactions will be used in an automated fashion. An example would be measuring changes in the amounts of the adenine nucleotides brought about in a measured interval of time by NDP kinase as determined after separation of the nucleotides by chromatographic procedures. Alternatively the effect of the compounds on nucleotide pools intracellularly can also be evaluated by preparing cell extracts and fractionating followed by HPLC. Also compounds that affect the oligomeric state of the kinase (determined by gel filtration methods) or its localization (electron microscopy) and association with other mitochondrial components can be readily designed. Screening for effects on mitochondrial function can be determined with end points for example being electrochemical potential gradient, oxidative phosphorylation, electron transport, oxygen uptake and mitochondrial transition pore activity.

Identifying Kinase Modulators for the Treatment of Several Diseases:

The invention relates to a method for treating cancer, viral infection, neurodegenerative disorders, insulin dependent diabetes mellitus and its related disorders, obesity, and mitochondrial related diseases in a subject afflicted with said disorder comprising administering to the subject an effective therapeutic amount of an adenylate kinase modulator. An adenylate kinase modulator is a compound that modifies (1) the activity of the enzyme and or its oligomerisation state (2) the adenine nucleotide pool regulated by this kinase (3) the location of the enzyme in the mitochondria or its ability to interact with other mitochondrial components (4) the energy transmitting function of the enzyme and its ability to connect the cytosol and mitochondrial communication and/or (5) fluxes in and out of the mitochondria.

Existing or specially designed combinatorial libraries will be screened for molecules that modulate the adenylate kinase system. Alternatively compounds could be rationally designed based on the know x ray structural information and the mechanism of action. Compounds of therapeutic interest could be identified as those that affect the enzymatic activity of adenylate kinase, its oligomeric state, nucleotide pools, its association with mitochondrial components, or it's effect on mitochondrial function among other parameters.

For identifying compounds that affect the enzymatic activity of adenylate kinase the above referred to enzymatic assay reactions will be used in an automated fashion. An example would be measuring changes in the amounts of the adenine nucleotides brought about in a measured interval of time by adenylate kinase as determined after separation of the nucleotides by chromatographic procedures. Alternatively the effect of the compounds on nucleotide pools intracellularly can also be evaluated by preparing cell extracts and fractionating followed by HPLC. Also compounds that affect the oligomeric state of the kinase (determined by gel filtration methods) or its localization (electron microscopy) and association with other mitochondrial components can be readily designed. Screening for effects on mitochondrial function can be determined with end points for example being electrochemical potential gradient, oxidative phosphorylation, electron transport, oxygen uptake and mitochondrial transition pore activity.

A glycerol kinase modulator is a compound that modifies (1) the activity of the enzyme and or its oligomerisation state (2) the substrates and products regulated by this kinase (3) the location of the enzyme in the mitochondria or its ability to interact with other mitochondrial components (4) the energy transmitting function of the enzyme and its ability to connect the cytosol and mitochondrial communication and/or (5) fluxes in and out of the mitochondria.

Existing or specially designed combinatorial libraries will be screened for molecules that modulate the glycerol kinase system. Alternatively compounds could be rationally designed based on x ray structural information and the mechanism of action of the enzyme. Compounds of therapeutic interest could be identified as those that affect the enzymatic activity of glycerol kinase, its oligomeric state, substrate and product pools, its association with mitochondrial components, or its effect on mitochondrial function among other parameters.

For identifying compounds that affect the enzymatic activity of glycerol kinase the above referred to enzymatic assay reactions or modifications of them will be used in an automated fashion. An example would be measuring changes in the amounts of products brought about in a measured interval of time by glycerol kinase as determined after separation of the nucleotides by chromatographic procedures. Alternatively the effect of the compounds on substrate and product pools intracellularly can also be evaluated by preparing cell extracts and fractionating followed by HPLC. Also compounds that affect the oligomeric state of the kinase (determined by gel filtration methods) or its localization (electron microscopy) and association with other mitochondrial components can be readily designed. Screening for effects on mitochondrial function can be determined with end points for example being electrochemical potential gradient, oxidative phosphorylation, electron transport, oxygen uptake and mitochondrial transition pore activity.

A hexokinase modulator is a compound that modifies (1) the activity of the enzyme and or its oligomerisation state (2) the adenine nucleotide pool regulated by this kinase (3) the location of the enzyme in the mitochondria or its ability to interact with other mitochondrial components (4) the energy transmitting function of the enzyme and its ability to connect the cytosol and mitochondrial communication and/or (5) fluxes in and out of the mitochondria.

Existing or specially designed combinatorial libraries will be screened for molecules that modulate the hexokinase system. Alternatively compounds could be rationally designed based on the known x ray structural information and the mechanism of action. Compounds of therapeutic interest could be identified as those that affect the enzymatic activity of hexokinase, its oligomeric state, nucleotide pools, its association with mitochondrial components, or its effect on mitochondrial function among other parameters.

For identifying compounds that affect the enzymatic activity of hexokinase the above referred to enzymatic assay reactions will be used in an automated fashion. An example would be measuring changes in the amounts of the adenine nucleotides brought about in a measured interval of time by hexokinase as determined after separation of the nucleotides by chromatographic procedures. Alternatively the effect of the compounds on nucleotide pools intracellularly can also be evaluated by preparing cell extracts and fractionating followed by HPLC. Also compounds that affect the oligomeric state of the kinase (determined by gel filtration methods) or its localization (electron microscopy) and association with other mitochondrial components can be readily designed. Screening for effects on mitochondrial function can be determined with end points, for example, being electrochemical potential gradient, oxidative phosphorylation, electron transport, oxygen uptake or mitochondrial transition pore activity.

The modes of administration for these compounds include, but are not limited to, oral, transdermal, or parenteral (e.g., subcutaneous, intramuscular, intravenous, bolus or continuous infusion). The actual amount of drug needed will depend on factors such as the size, age and severity of disease in the afflicted individual. For this invention the NDP kinase compound will be administered at dosages and for periods of time effective to reduce, ameliorate or eliminate the symptoms of the disease. Dose regimens may be adjusted for purposes of improving the therapeutic or prophylactic response of the compound. For example, several divided doses may be administered daily, one dose, or cyclic administration of the compounds to achieve the desired therapeutic result. Agents that improve the solubility of these compounds could also be added.

One skilled in the art could prepare modulators suitable as substrates with the kinases described above. Suitable substrates would include derivatives of the corresponding substrate, e.g., derivatives or analogs of adenosine, glycerol, hexoses and diphospho nucleosides. Modification of these substrates can be effected under standard techniques known to those skilled in the art. An example of such a modification would be replacement of a oxygen atom with another heteroatom, replacement of a carbon within the carbon backbone with a heteroatom, replacement of one or more hydrogen atoms with substitutents, e.g., an alkyl group, a halide, or an ester. Examples of suitable compounds, modulators, include but are not limited to bepridil, verapamil, nifedipine, nisoldipine, bronchodilators, e.g. theophylline, semecarpus anacardium nut extract, 2-dioxy-D-glucose, antioxidants, e.g. vitamin E, purines, such as caffeine or theophylline, or dipyridamole, papaverine, or cyclamidomycin (desdanine).

The modulators can be formulated with one or more adjuvants and/or pharmaceutically acceptable carriers according to the selected route of administration. The addition of gelatin, flavoring agents, or coating material can be used for oral applications. For solutions or emulsions in general, carriers may include aqueous or alcoholic/ aqueous solutions, emulsions or suspensions, including saline and buffered media. Parenteral vehicles can include sodium chloride, potassium chloride among others. In addition, intravenous vehicles can include fluid and nutrient replenishers, electrolyte replenishers among others.

Preservatives and other additives can also be present. For example, antimicrobial, antioxidant, chelating agents, and inert gases can be added (see, generally, Remington's Pharmaceutical Sciences, 16th Edition, Mack, (1980)).

The contents of all references, pending patent applications and published patent applications, cited throughout this application, including those referenced in the background section, are hereby incorporated by reference. It should be understood that the models or assays used throughout the examples are accepted models and that the demonstration of efficacy in these models is predictive of efficacy in humans.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating an energy related associated state, comprising administering to a subject an effective amount of an energy kinase modulator, such that an energy related associated state is treated.

2. The method of claim 1, wherein said energy related associated state is related with an energy related associated state is a state associated with energy impairment.

3. The method of claim 1, wherein said energy related associated state is related with an energy related associated state is a state associated with energy transmission.

4. The method of claim 1, wherein said energy related associated state is related with an energy related associated state is a state associated with energy consumption.

5. The method of claim 1, wherein said energy kinase modulator is bepridil, verapamil, nifedipine, nisoldipine, theophylline, semecarpus anacardium nut extract, 2-dioxy-D-glucose, vitamin E, caffeine, dipyridamole, papaverine, or cyclamidomycin (desdanine).

6. The method of claim 1, wherein said energy related state is associated with a neurodegenerative disorder, insulin dependent diabetes mellitus or its related disorders, obesity, mitochondrial related diseases or a viral infection.

7. The method of claim 1, wherein the subject is a mammal.

8. The method of claim 7, wherein the subject is a human.

9. A method for modulating an energy transmitting kinase in a subject, comprising administering to a subject an effective amount of an energy transmitting kinase modulator, such that an energy transmitting kinase is modulated.

10. The method of claim 9, wherein said energy transmitting kinase is associated with energy impairment.

11. The method of claim 9, wherein said energy transmitting kinase is associated with consuming energy.

12. The method of claim 9, wherein said energy transmitting kinase is associated with transmitting energy.

13. The method of claim 9, wherein said energy transmitting kinase is associated with a neurodegenerative disorder, insulin dependent diabetes mellitus or its related disorders, obesity, mitochondrial related diseases or a viral infection.

14. The method of claim 9, wherein the subject is a mammal.

15. The method of claim 14, wherein the subject is a human.

16. A method of treating an energy related associated state, comprising administering to a subject an effective amount of an energy kinase modulator, such that an energy related associated state is treated, provided said energy related associated state does not involve creatine kinase.

17. The method of claim 16, wherein said energy related associated state is related with an energy impairing kinase.

18. The method of claim 16, wherein said energy related associated state is related with an energy transmitting kinase.

19. The method of claim 16, wherein said energy related associated state is related with an energy consuming kinase.

20. The method of claim 16, wherein said energy kinase modulator is

21. The method of claim 16, wherein said energy related state is associated with a neurodegenerative disorder, insulin dependent diabetes mellitus or its related disorders, obesity, mitochondrial related diseases or a viral infection.

22. The method of claim 16, wherein the subject is a mammal.

23. The method of claim 22, wherein the subject is a human.
